(19) United States
(12) Patent Application Publication
(10) Pub. No.: US 2025/0380939 A1
(43) Pub. Date: Dec. 18, 2025

(54) LATERAL RETRACTOR SYSTEMS AND METHODS

(71) Applicant: GLOBUS MEDICAL, INC., AUDUBON, PA (US)

(72) Inventors: Andrew Hartman, San Diego, CA (US); Byron Reimhofer, San Diego, CA (US); Michael Serra, San Diego, CA (US)

(21) Appl. No.: 18/743,285

(22) Filed: Jun. 14, 2024

(51) Int. Cl. A61B 17/02; A61B 17/00

(52) U.S. Cl. CPC A61B 17/025; A61B 17/0206; A61B 2017/00367; A61B 2017/0256

(57) ABSTRACT

A lateral retractor system has components and features to permit control of each retractor arm so as to articulate independently. The system likewise includes the ability to lock one arm in place and articulate only the other, as well as the ability to toggle or switch between modes, including the mode where the arms articulate together, equally.

### FIELD

This disclosure relates to devices useful in spinal surgery, in general, and to lateral retractors, in particular.

### BACKGROUND

Lateral retractors currently used in conjunction with certain spinal operations may benefit from additional functionality and flexibility when in use For example, there may be situations where flexibility in how a medical professional causes movement of cranial and caudal arms of the retractor would benefit operations, or expand the potential applications where a lateral retractor is useful.

It would be advantageous to foster some of the foregoing benefits with a corresponding lateral retractor system.

### SUMMARY

In one possible implementation of the disclosure, a lateral retractor system has a pair of arms, that is, a cranial arm and a caudal arm, each arm pivotably secured to articulation axes. The arms have portions extending from such articulation axes so that inward movement or squeezing of proximal portions toward a central longitudinal axis causes outward movement of corresponding distal portions to perform lateral retraction. The arms can be locked by suitable locking mechanisms which, in one implementation, lock a pinion on one or both of the arms, such pinion otherwise rotating along the track of a cranial-caudal rack. Locking one or the other of the pinions associated with arms with the locking mechanism prevents rotation of the corresponding arm around the articulation axis.

In another potential implementation, a locking foot is used to arrest rotation of the pinion relative to the rack, the locking foot being moveable into and out of engagement with the pinion by various means, including depressing of a user accessible button.

In still another potential implementation, the locking mechanisms makes use of a pair of split bearings to lock corresponding arms relative to each other. The split bearings are coupled to the inner ends of driveshafts which extend generally transversely to a central longitudinal axis. Using this configuration of split bearings permits locking mechanisms to arrest rotation around pivot points of respective locking arms. Arresting of the pivot point locks that arm, but the split bearings permit the other arm to continue to rotate and therefore move in relation to the locked arm.

In accordance with these or other implementations or variations, a lateral retractor system may have features and components that make up an equal bilateral retraction mechanism. Such equal bilateral retraction mechanism functions to give medical professionals the ability to selectively set the arms between independent movement relative to each other, or equal inward and outward movement relative to each other. Toggling into or actuating the equal bilateral retraction mode causes articulation by equal amounts because movement of one arm is tied to movement of the corresponding arm. In one possible implementation, such equal bilateral retraction mechanism uses face gears interconnecting the arms selectively by means of a clutch. When the clutch is disengaged, the arms may move independently of each other; however, when the clutch is engaged, such engagement causes equal inward and outward movement of the arms relative to each other.

In another potential implementation, the equal bilateral retraction mechanism makes use of a knurled clutch which couples two components, associated with each of the arms, such as the split bearings described previously. Engagement of the knurled clutch on corresponding knurled engagement surfaces of the split bearing would cause motion of one of the arms to induce corresponding motion in the other of the arms, resulting in equal bilateral retraction, meaning equal inward and outward motion of the arms relative to each other.

### BRIEF DESCRIPTION OF THE DRAWINGS

A more complete understanding of the present invention, and the attendant advantages and features thereof, will be more readily understood by reference to the following detailed description when considered in conjunction with the accompanying drawings, wherein:

FIG. 1 is a top plan view of one possible implementation of a lateral retractor system according to the present disclosure;

FIG. 2 is an isometric partial view of a pair of locking mechanisms of the implementation of FIG. 1;

FIG. 3 is an isometric, exploded view of one implementation of a locking mechanism of the implementations of FIGS. 1 and 2;

FIGS. 4A-4D are successive, side-elevational, cut away views of a possible implementation of the locking mechanism or FIGS. 1-3;

FIG. 5 is an exploded, isometric view of another possible implementation of a locking mechanism for an implementation of the lateral retractor system of FIGS. 1-2;

FIGS. 6A-6C are successive, side-elevational, cut away views of the implementation of the locking mechanism shown in FIG. 5;

FIG. 7 is an isometric, cut away view of another potential implementation of a locking mechanism for a lateral retractor system;

FIG. 8 is an enlarged, isometric, sectional view of components of a lateral retractor system according to the present disclosure;

FIG. 9 is an isometric, sectional view of the components of FIG. 8 for a possible implementation of a lateral retractor system;

FIG. 10 is an isometric view of still other components of another potential implementation of a lateral retractor system according to the present disclosure;

FIG. 11 is a top, plan view of another possible implementation of a lateral retractor system according to the present disclosure;

FIG. 12 is a top, plan view of the implementation shown in FIG. 11, with the left arm fully opened;

FIG. 13 is an isometric, partial, cut away view of another possible implementation of a locking mechanism for a lateral retractor system according to the present disclosure;

FIG. 14 is a side, sectional view of components of a locking mechanism according to the implementation of FIG. 13;

FIG. 15 is an isometric, partial, cut away view of a locking mechanism according to the implementation of FIGS. 13-14;

FIG. 16 is a top, plan view of still another potential implementation of a lateral retractor system according to the present disclosure;

FIG. 17 is a side, elevational view of the implementation shown in FIG. 16 of a lateral retractor system;

FIG. 18 is a top plan, partial, cut away view of another potential locking mechanism for an implementation of a lateral retractor system, such as that of FIGS. 16 and 17;

FIGS. 19A-19B are isometric, successive views of components of the locking mechanism of FIGS. 16-18, showing components in two different positions;

FIG. 19C is a side, cross-sectional view of the locking mechanism of FIGS. 19A-19B;

FIG. 20 is an exploded, isometric view of components of implementations of a lateral retractor system according to the present disclosure; and

FIG. 21 is an enlarged, isometric view of certain of the components of the implementation of the lateral retractor system of FIG. 20.

### DETAILED DESCRIPTION

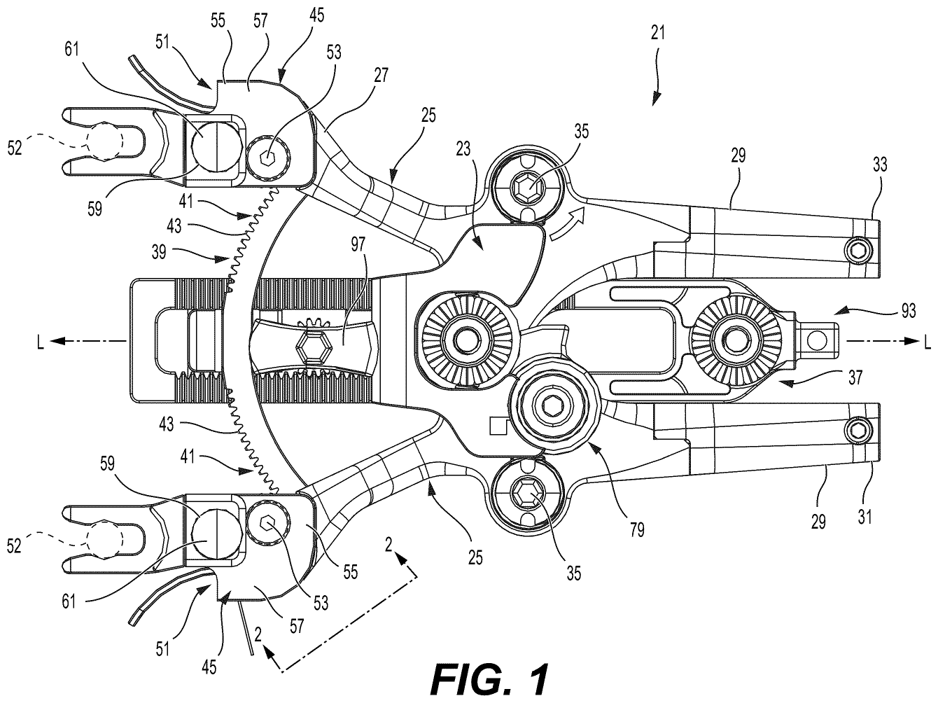

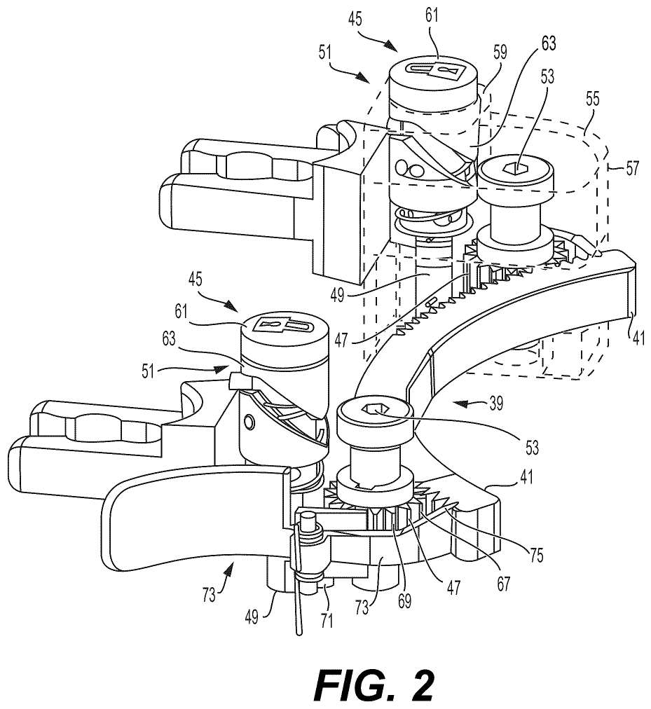

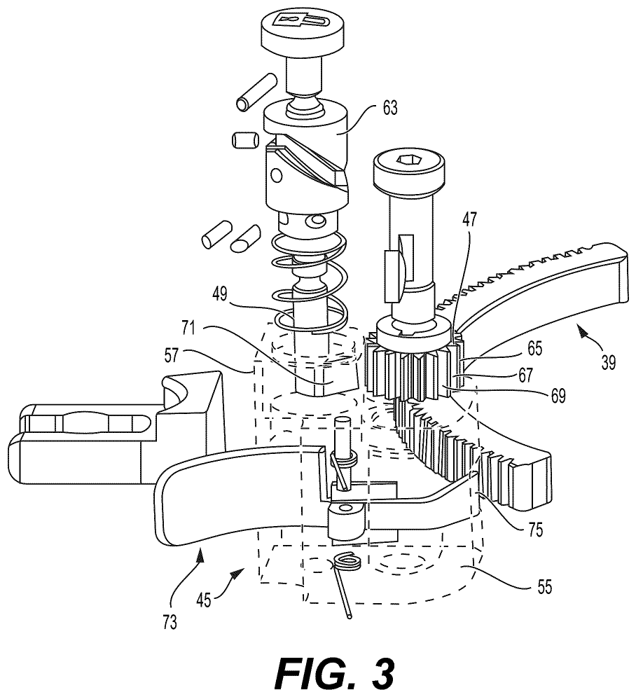

Referring now to the drawings and, in particular, to FIGS. 1-10, a lateral retractor system 21 includes components, to be disclosed herein, which enable at least three features and functions. First, the lateral retractor system 21 allows control of each of its arms 25, so that they can articulate independently. Secondly, the components to be described herein allow lateral retractor system 21 to lock one of the arms 25 in place and thereby move or articulate only the other arm. Thirdly, features of lateral retractor system 21 allow a user to toggle between modes where arms 25 articulate together equally, referred to as equal bilateral retraction or EBR, or have independent articulation, that is, not equal retraction as between the arms relative to each other. This last feature can be actuated from any relative position of arms 25 relative to each other.

Lateral retractor system 21 includes a central subassembly 23, to which a pair of the arms 25 are connected about respective, articulation axes 35. Articulation axes 35 are located in laterally spaced relation to each other relatively to a longitudinal axis L defined in system 21, thereby spacing arms 25 from each other to define a cranial arm 31 and a caudal arm 33. Extending outwardly in the proximal and distal directions from articulation axes 35, arms 25 include respective proximal and distal portions 27, 29. The relative location of proximal and distal portions 27, 29 are such that when system 21 is operated so that proximal portions 27 move inwardly towards longitudinal axis L, such movement causes corresponding outward movement of distal portions 29 of arms 25, such as may be required to perform lateral retraction on patient anatomy undergoing a spinal procedure.

A third central arm 37 translates axially through a given center point of retractor system 21, generally parallel to longitudinal axis L between cranial and caudal arms 31, 33. Retraction of cranial and caudal arms 31, 33 is controlled by an arched, rack-and-pinion mechanism connected to central subassembly 23, running between cranial and caudal arms 31, 33. In the illustrated implementation, the rack-and-pinion mechanism comprises a cranial-caudal rack 39 connected to subassembly 23 and extending arcuately and outwardly on both sides of longitudinal axis L, and a pair of pinions 47.

Pinions 47 rotate relative to rack 39 during articulation of arms 25, and are likewise operatively associated with corresponding locking mechanisms 47, which mechanisms can selectively arrest rotation of corresponding pinions 47, as disclosed herein. Rack 39 terminates in opposite rack ends 41 located proximate to proximal portions 29 of arms 25. Rack ends 41 have rack teeth 43 formed thereon. The structural arrangement of the rack-and-pinion mechanism relative to arms 25 allow its operation independently of how central arm 37 is driven.

Each arm 25 contains a housing proximate to the intersection of arms 25 and rack 39. Locking mechanisms 45 may be actuated to arrest or lock movement of the corresponding arm to which locking mechanism 45 is connected. Each of the locking mechanisms 45 are operatively connected to a corresponding one of the pinions 47. In this implementation, such operative connection includes a locking foot 49 which is selectively actuatable by the user to engage the aforementioned pinion 47 or, alternately, to directly engage an opposing location of rack 39. By locking movement of pinion 47, since pinion 47 is rotatably engaged in rack teeth 43, the result is that movement of the corresponding arm 25 is arrested or otherwise prevented. Accordingly, in view of the foregoing, with a locking mechanism 45 on each of the arms 25, actuation of one locking mechanism will allow that arm to be controlled independently of the other arm and vice versa. Furthermore, both locking mechanisms could be actuated at the same time to arrest movement or articulation of arms 25 relative to each other.

In general terms, then, a structure such as locking foot 49 or similar stop mechanism is movable into the teeth or cogs of pinion 47 by actuating or toggling locking mechanism 45 between locked and unlocked states, thereby arresting rotation of corresponding pinion 47 relative to rack teeth 43 and locking the corresponding arm.

In one possible implementation, a button 61 is disposed in a bore 59 defined on outer surface 57 of housing 55. A barrel cam 63 is rotatably received in bore 59 and operatively connected to a pressable button 61. Barrel cam 63, in turn, is connected to engage locking foot 49 against pinion 47. In response to user actuation of button 61, barrel cam 63 moves to either engage or disengage locking foot 49 against pinion 47.

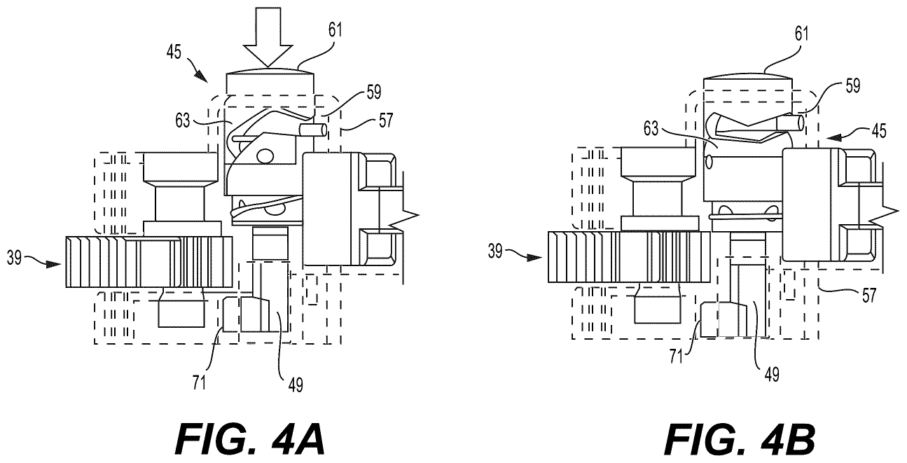

Referring more particularly to FIGS. 4A, 4B, 4C, and 4D, in one possible implementation, locking mechanism 45 is actuated by the pressing button 61 in the direction shown by the arrow in FIG. 4A, which rotates barrel cam 63 from the position shown in FIG. 4A to an intermediate position shown in FIG. 4B. Suitable spring biasing returns button 61 upwardly but barrel cam 63 assumes a third position shown in FIG. 4C, in which locking foot 49 engages a suitable cog or tooth of pinion 47 as shown to lock pinion 47. Pushing button 61 again, as shown by the arrow in FIG. 4C, returns locking mechanism 45 and its associated barrel cam 63 to the unlocked position shown in FIG. 4D, identical to that shown in FIG. 4A.

The interacting and operatively engaging portions of pinion 47 and locking foot 49 may assume a variety of configurations suitable for arresting the relative motion of pinion 47 along rack 39. In the illustrated implementation, pinion 47 has pinion teeth 67 with female portions 69 defined between pinion teeth 67 on cylindrical engagement surface 65 of pinion 47. For its part, locking foot 49 has a shaft from which an engagement portion extends at right angles to terminate in an engagement edge 71. Engagement portion and its engagement edge 71 are located and sized to engage one of the female portions 69 between upper edges of pinion teeth 67. Locking foot 49 is suitably secured within housing 55 against disengagement from transverse forces relative to rack 39, such that engagement of engagement edge 71 into the corresponding female portion 69 arrests movement not only of pinion 47 itself relative to rack 39, but also arrests movement of the corresponding arm 25, thereby locking such arm.

In the illustrated embodiment, locking foot 49 as shown in FIG. 4A starts above pinion 47 and drives down to lock the corresponding pinion and arm, as shown in FIG. 4C. Other implementations are possible, such as configuring locking foot 49 to start below rack 39 and, by suitable actuation of barrel cam 63, causing foot 49 to move up into locked position against pinion 47.

Lateral retractor system 21 is able to be driven by moving or squeezing proximal portions 27 towards each other by means of a suitable handle 52, but may likewise be operated by a separate tool couplable to retractor system 21 by means of a driver 53. Both such means of articulating the proximal portions 27 in order to cause inward and outward movement of cranial and caudal arms 31, 33 may be suitably connected through an attachment interface 51 disposed on each of the ends of proximal portions 27, as shown. Attachment interface 51 includes suitable structures for interfacing with any suitable handle, whether manually or automatically actuated, and a suitable driver 53 may take a variety of forms, such as a hex connection, either female or male.

Each of the arms 25 has associated with it at its proximal ends a pawl 73 located in housing 55 and having a distal end 75 extending distally. Distal end 75 of pawl 73 incrementally engages rack teeth 43 as arms 25 are urged together or apart, and as pinion 47 moves relative to rack teeth 43 of rack 39. Pawl 73 and its distil end 75 are connected by a suitable distance from the engagement edge 71 of locking foot 49 to maintain relative position therebetween and such positioning maintains alignment between edge 71 and corresponding female portions 69 of pinion 47. In this way, as arms 25 move, and as pinion 47 travels relative to rack 39, pawl 73 is located such that engagement edge 71 of locking foot 49 is always able to engage female portions 69 to lock the arms regardless of the position of pinion 47 relative to rack 39.

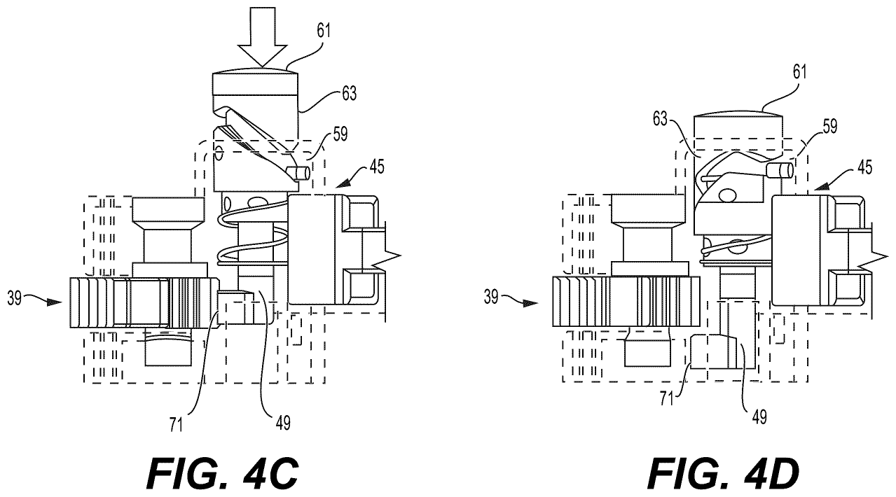

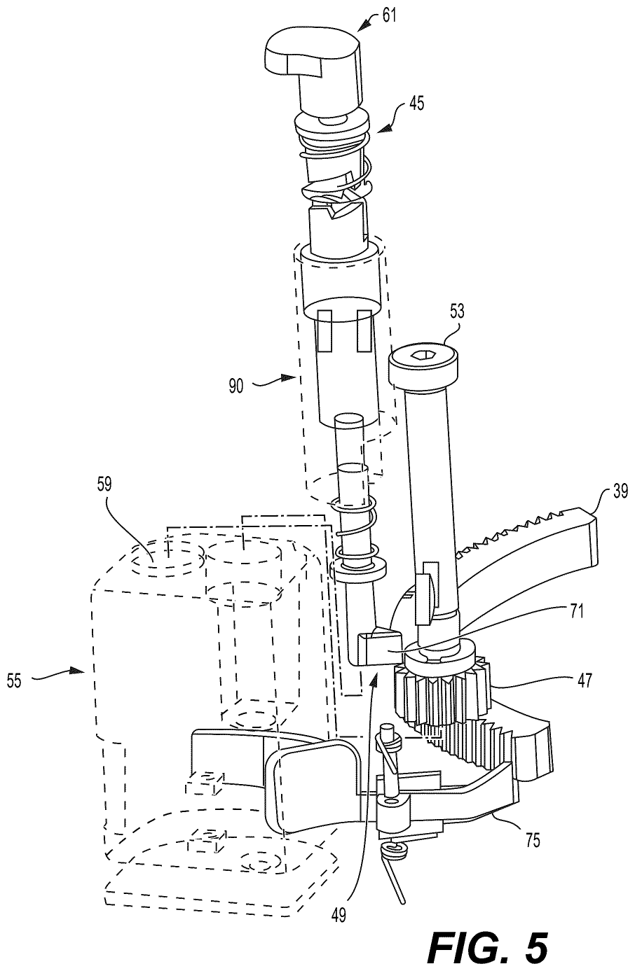

In another suitable implementation shown in FIGS. 5-6A-C, a pin-click mechanism 50 is used in locking mechanism 45 instead of barrel cam 63. Similar to the previous implementation, a user depresses button 61 of locking mechanism 45 which, in turn, moves locking foot 49 into or out of engagement with pinion 47. Suitable springs, keys, and cylindrically oriented parts, such as those associated with a mechanism found in a retractable ballpoint pen, are used to move locking foot 49 between engagement and disengagement, in the same matter as the tip of the ball point pen is extending and retracted relative to a writing surface. FIG. 6A shows locking mechanism 45 in the unlocked position, at which point user pushes button 61 downward as shown. After clearing stop members associated with pen-click mechanism 50 on housing 55, a lower cam releases and is held down by a corresponding stop member, as shown in FIG. 6B, to lock corresponding pinion 47. A further actuation as shown by the arrow in FIG. 6B returns the came to the position shown in FIGS. 6A and 6C, in which stop members sit in slots associated with the cam, thereby unlocking mechanism 50, which in turn frees pinion 47 to move relative to rack 39 once again.

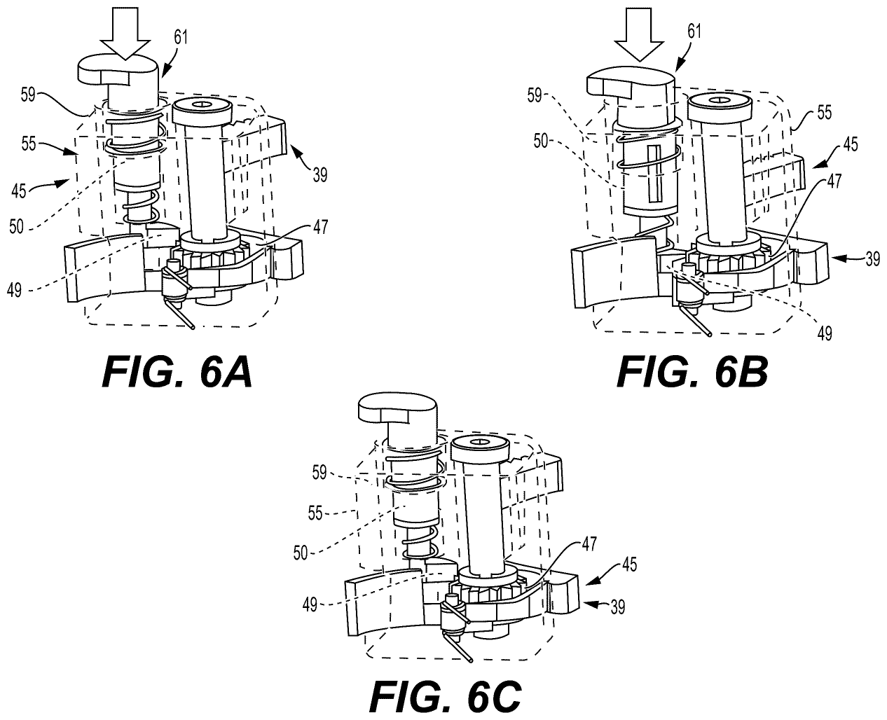

Another possible implementation of locking mechanism 45 is shown with reference to FIG. 7. A user-accessible locking screw 77 may be suitably rotated to advance or retract locking foot 49 relative to opposing female portions 69 of pinion teeth 67 on pinion 47. Rotation of locking screw 77 relative to locking foot 49 in a first direction advances locking foot 49 toward pinion 47, and rotation in a second direction disengages the two components from each other to unlock them.

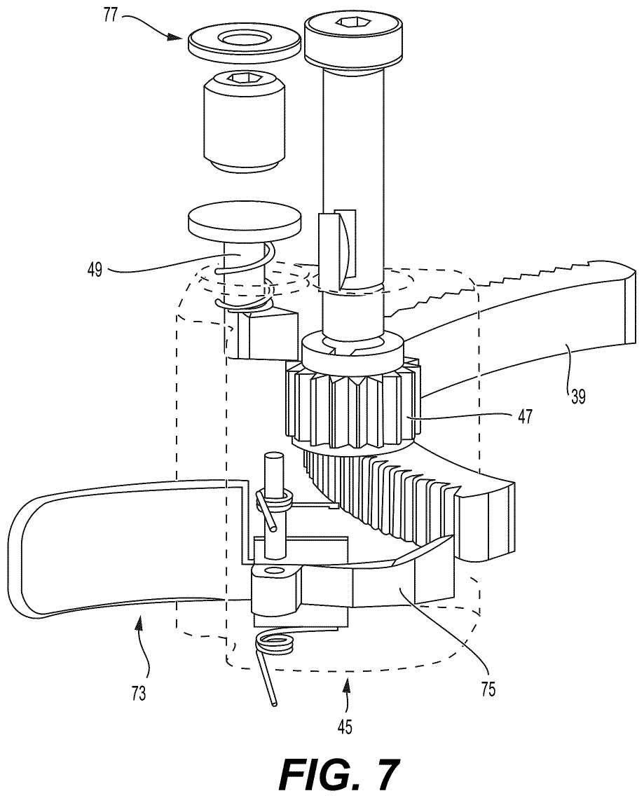

Referring now more particularly to FIGS. 8-10, lateral retraction system 21 has components and features such that motion of each arm 25 can be selectively coupled, so that the opening of one arm causes an equal opening of the other arm. In one suitable implementation, the foregoing functionality may be achieved by incorporating a selectively actuatable geared mechanism at the interface between the two arms 25, such as around a fixed center point. One arm has a fixed gear built into its body, while the other arm has a gear that is free to rotate about a shaft on such other arm, such latter gear being driven by the former gear when the arm is in the unlocked state.

In the particular implementation illustrated in FIGS. 8-10, an equal, bilateral retraction mechanism 79 is secured to central subassembly 23 and has at least one gear 81 operatively connected to each of arms 25. Gears 81 are mounted so as to be arrestable to actuation of bilateral retraction mechanism 79, which connects motion of one of the arms 25 to the other arm 25, thereby moving the arms equally in response to user-initiated movement of proximal portions 27 of arms 25. At least one of gears 81 is configured to be a face gear 83, and equal bilateral retraction mechanism 79 makes use of a user-actuatable clutch 85 to selectively engage face gear 83.

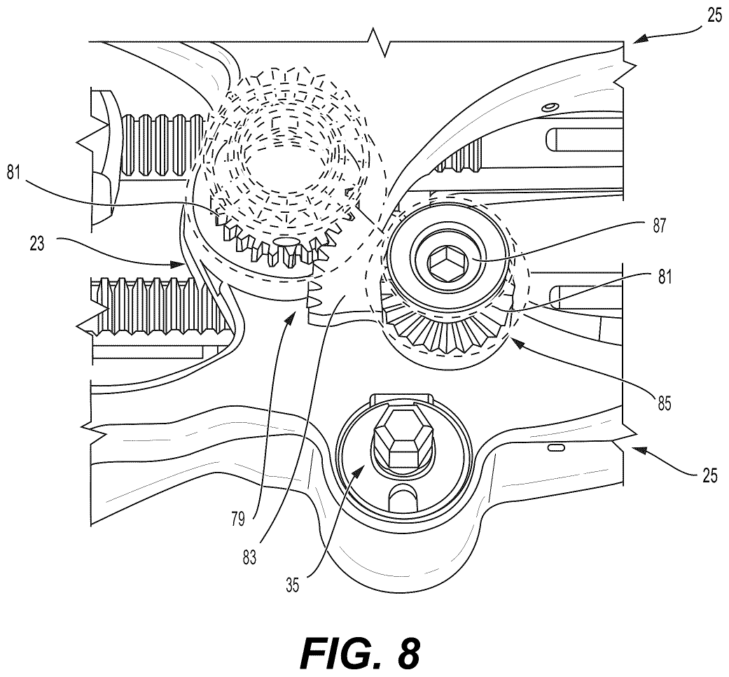

Clutch 85 has a clutch plate 87 which may be urged to engage face gear 83. Face gear 83 includes an arcuate, flanged portion extending radially outwardly which intermeshes with another of the gears 81 rotatably mounted about the other of the arms 25, as shown in FIGS. 8-9 in particular. By advancement of clutch 85 in the downward direction as orientated in FIG. 9, a corresponding axially mounting gear 81 engages face gear 83 below it. Such engagement is transmitted to the radially extending flange portion of face gear 83, and such flange portion, in turn, transmits motion of one arm 25 to the other arm 25 through the interconnection of gears 81 and flange portion of face gear 83. Gears 81 are sized such that the foregoing interconnections cause both arms to have equal relative movement, including equal bilateral retraction.

Clutch 85 in the illustrated implementation may be driven through a threaded or otherwise advanceable hex connection or any suitable means to move the upper one of gears 81 into operative contact with the lower one of gears 81, that is, face gear 83. Equal bilateral retraction mechanism 79 may be engaged or disengaged, that is, toggleable, at any relative position of arms 25, whether arms are fully closed or in any state of open. As described above, clutch 85 drives a corresponding bolt down to engage face gear 83 interconnecting gears 81 on respective arms 25, whereas backing off the thread clutch disengages the overlying gear 81 from engagement with remaining gears 81, such that the gears skip over each other without coupling motion of one arm to the other.

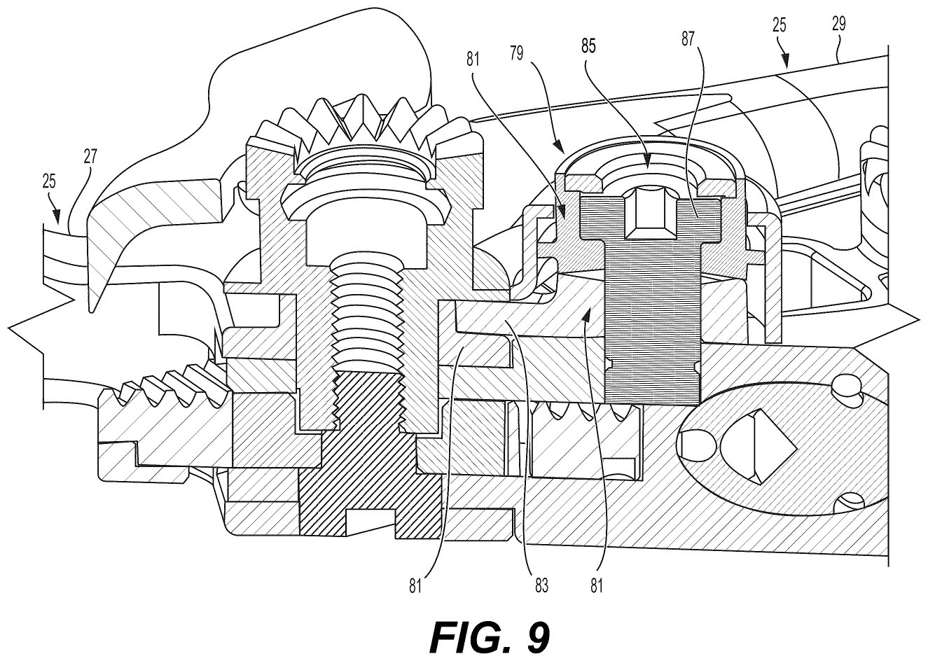

Referring more particularly to FIG. 10, central arm 37 comprises a rack-and-pinion subassembly 89 which has associated therewith a central arm pinion 91. Rack-and-pinion subassembly 89 is selectively advanceable by movement of central arm pinion 91 relative to the underlying rack to advance and withdraw a distal arm end 93 of central arm 37. Central arm pinion 91 is suitably connected to a central arm driveshaft 95 terminating in a handle 97 to effectuate advancement and withdrawal of central arm 37, such as toward or away from an operative area of a patient undergoing corresponding surgery. The foregoing driving mechanism for central arm 37 may be unconnected to the motion of cranial and caudal arms 31, 33 thereby permitting selective movement of central arm 37 independently of movement of cranial and caudal arms 31,33.

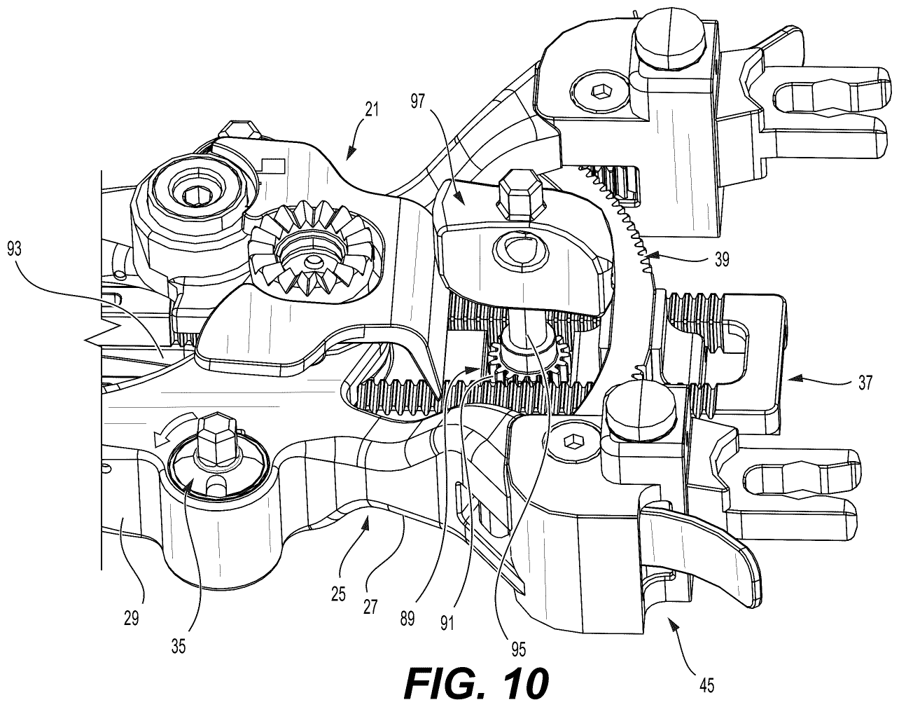

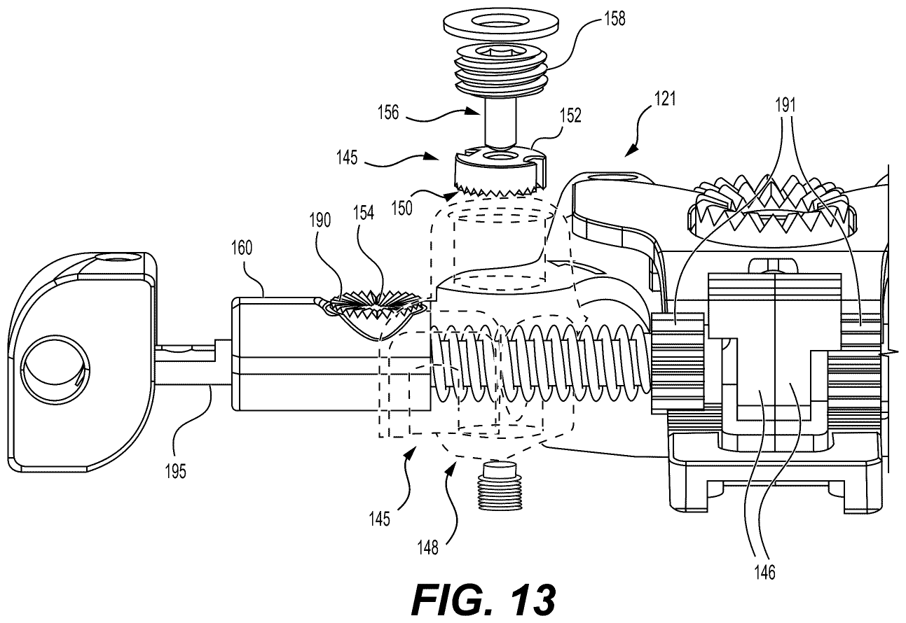

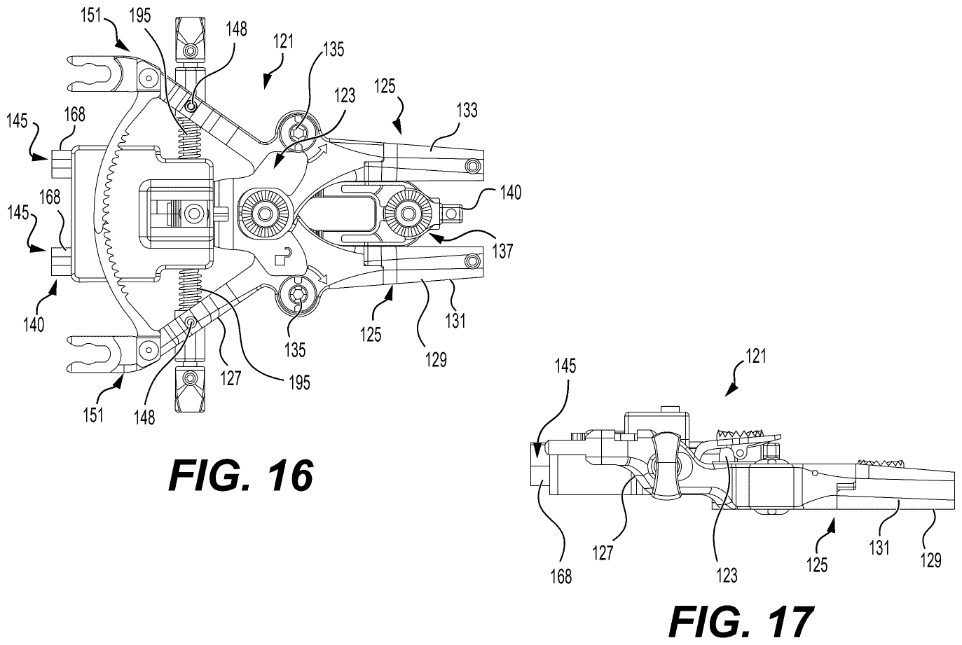

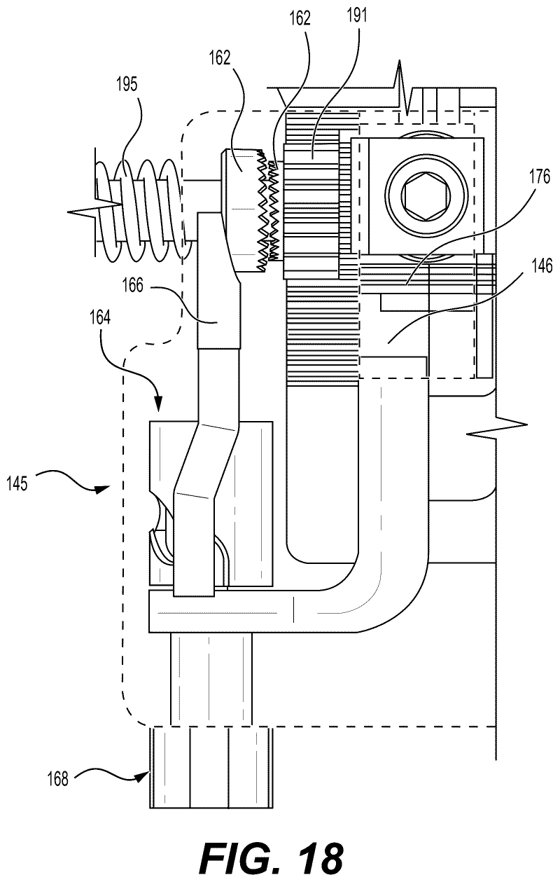

Referring now more particularly to FIGS. 11-21, another implementation of a lateral retractor system 121 is shown and described, with similar components referenced with similar reference numerals preceded by a numeral 1, meaning such similar references are in a one hundred series as compared to reference numbers in FIGS. 1-10. Accordingly, as in the embodiment of FIGS. 1-10, lateral retractor system 121 includes a central subassembly 123 having a pair of arms 125 with respective proximal and distal portions 127, 129. Arms 125 are connected to subassembly 123 relative to a central longitudinal axis L and in space relation to each other, so that the two arms 125 define a cranial arm 131 and a caudal arm 133.

Connection of arms 125 is made through respective articulation axes 135 located such that inward movement of proximal portions 127 of arms 125 towards central longitudinal axis L causes outward movement of distal portion 129 of respective arms 125.

Central arm 137 has opposite lateral sides 138 and opposite proximal and distal ends 140. Central arm 137 is movably connected to central subassembly 123 and selectively advanceable and retractable longitudinally to move the distal end relative to a patient anatomy undergoing a spinal procedure. Central arm rack 189 extends in a longitudinal direction in the same manner as longitudinal axis L.

Central arm rack 189, in this embodiment, includes additional or different features from central arm rack 89 of the previous embodiment. Central arm rack 189 is configured so as to be connectable to a pair of driveshafts 195 extending transversely from lateral sides 138 of central arm 137 in relations to longitudinal axis L. As such, driveshafts 195 define respective inner and outer driveshaft ends 142, 144, the inner driveshaft ends 142 being rotatably connected to central arm rack 189 at respective separate locations by means of central arm pinions 191. By virtue of such connections, central arm 137 may be driven distally or proximally using either of driveshafts 195. Rotation of driveshafts 195 rotates corresponding central arm pinions 191 which are suitably fixed on central subassembly 123 so that such rotation of the pinion causes distal and proximal movement of central arm 123.

Unlike the previous embodiment, two, split bearings 146 are disposed on central arm 137 and connected at its proximal end to central arm rack 189. Split bearings 146 are disposed relative to central rack 189 to translate longitudinally relative to each other and relative to central rack 189 when arms 125 are moved or otherwise articulated. Bearings 146 are housed, disposed, or otherwise sit within central rack 189. However, bearings 146 do not influence the motion of central arm 137. Bearings 146 may be operatively located within housing 155, which housing 155, in turn, is connected to central subassembly 123.

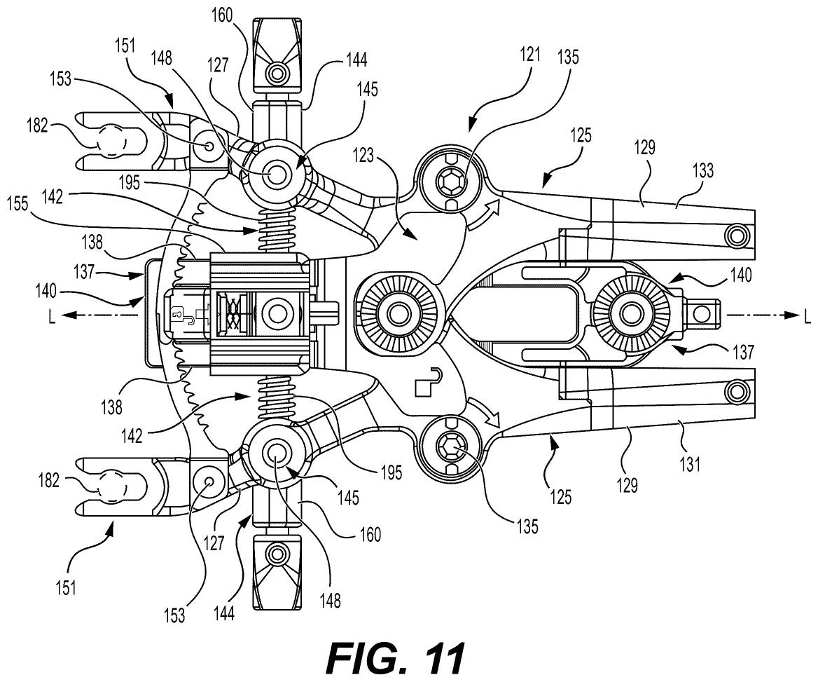

Outer driveshaft ends 144 are pivotally connected at pivot points 148 on proximal portions 127 of arms 125, whereby movement of one of the arms 125 relative to the other arms 125 translates the corresponding one of the bearings 146 relative to the other one. This relative movement of bearings 146 is shown with reference to FIG. 12, where the opening of arm 125, in this case caudal arm 133, has caused movement of its corresponding driveshaft 195 in the direction of longitudinal axis L, as well as movement of its corresponding pinion 191, which pinion is operatively associated with a corresponding one of split bearings 146. As illustrated, upon opening of caudal arm 133. the corresponding split bearing 146 translates, along with its underlying pinion 191, in the proximal direction relative to the corresponding components associated with cranial arm 131.

Lateral retractor system 121, similar to previous embodiment, includes a pair of locking mechanisms 145, but such locking mechanisms are operatively connected to arms 125 in another manner in the implementation of FIGS. 11-21, as disclosed herein. Each of the locking mechanisms 145 are configured to lock a corresponding one of the pivot points 148 in this implementation, in response to user actuation, thereby arresting movement of the corresponding one of arms 125 independently of the other arm 125.

Referring to FIGS. 11-15 more particularly, locking mechanism 145 makes use of a pair of opposing, matable face gears 150 which are axially mounted relative to the corresponding axis of pivot points 148. The upper one of face gears 150, as shown in the orientation of FIGS. 11-15, is formed to be a free gear 152 keyed rotationally to its corresponding arm 125, and is free to rotate about the corresponding pivot point 148 when such arm is pivoted about such pivot point 148. The second of the face gears, in this case shown as the lower one in this orientation, may be a fixed gear 154 non-rotatably mounted relative to pivot point 148.

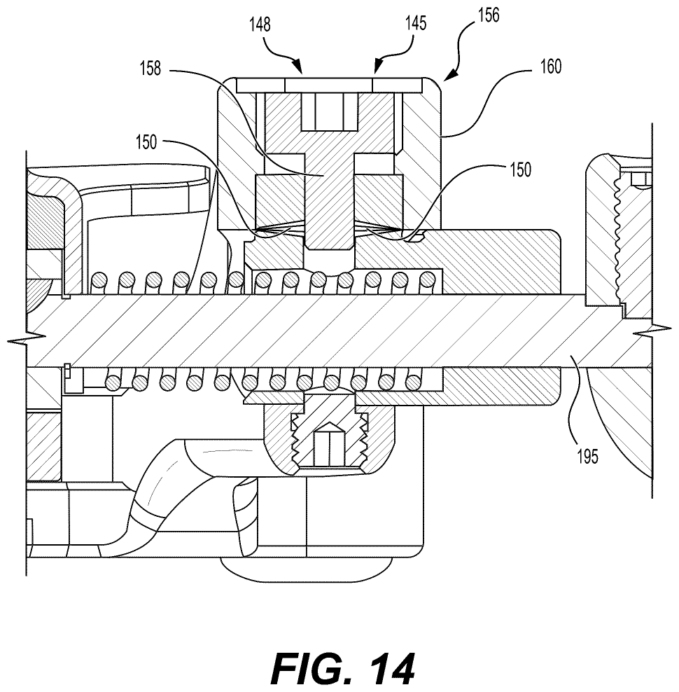

In such configuration, the opposing surfaces of gears 150 are mounted to be translatable between first and second positions, the first position corresponding to a disengaged position in which the opposing surfaces are in a spaced relationship, whereas the second position corresponds to engaged position in which the opposing surfaces are mated. FIG. 15 shows the first, disengaged position in which the opposing surfaces do not engage each other and so are free to skip over or rotate relative to each other, allowing the corresponding arm to rotate relative to pivot point 148.

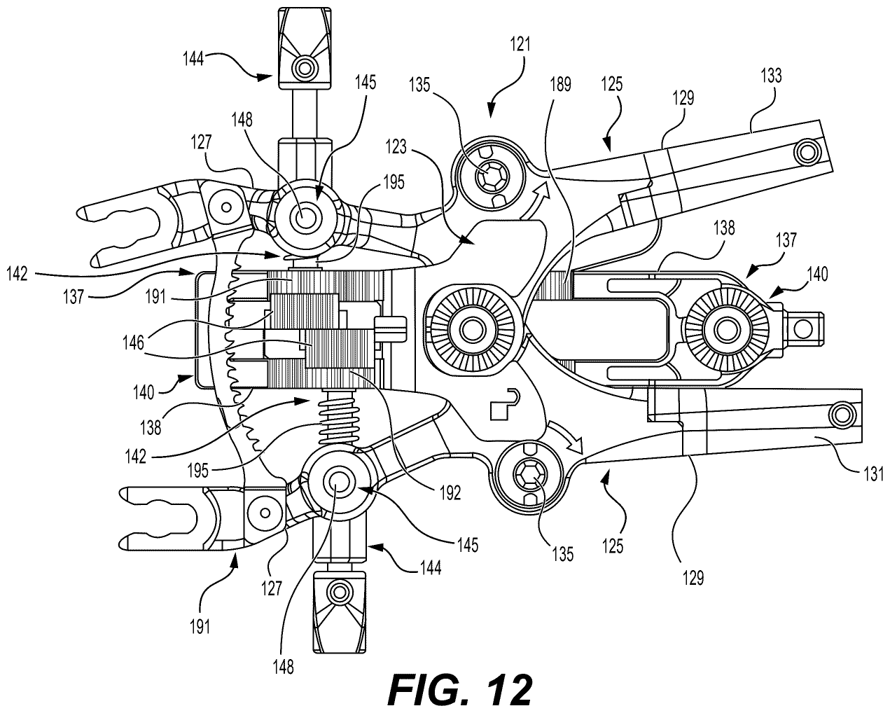

Locking mechanism 145 makes use of a clutch 156 to selectively move the pair of gears 150 between the disengaged and the engaged positions. In the illustrated implementation of FIGS. 13-15, clutch 156 makes use of a threaded set screw 158 which is rotatable in a first direction to move face gears 150 relatively toward each other to engage the locking mechanism 145, and rotatable in a second direction to move face gears 150 relatively away from each other, as shown in FIG. 15, to disengage the locking mechanism 145. Locking mechanisms 145 are suitably mounted relative to pivot points 148, such as with corresponding bushings 160 which are disposed to surround driveshafts 195 on respective proximal portions 127 of arms 125.

Operation of locking mechanism 145 in the implementation illustrated in FIGS. 13-15 is readily apparent from the foregoing description. Rotation about pivot point 148 occurs as a result of articulation of the corresponding arm 125, so locking such rotation will act to prevent retraction or other movement of the corresponding arm. Such locking of rotation about pivot points 148 occurs when face gears 150 and their opposing surfaces can be mated by suitable actuation of clutch 156. When not so mated, the opposing surfaces of face gears 150 have suitable separation so that they skip relative to each other or do not engage, thereby not impinging upon rotation of about pivot point 148 which normally occurs. In a non-locking state, locking mechanism 145 is keyed to arm 125 and may vertically skip up within arm 125 when face gear 154 rotates. Closing clutch 156 prevents the vertical skipping and locks locking mechanism 145 to face gear 154.

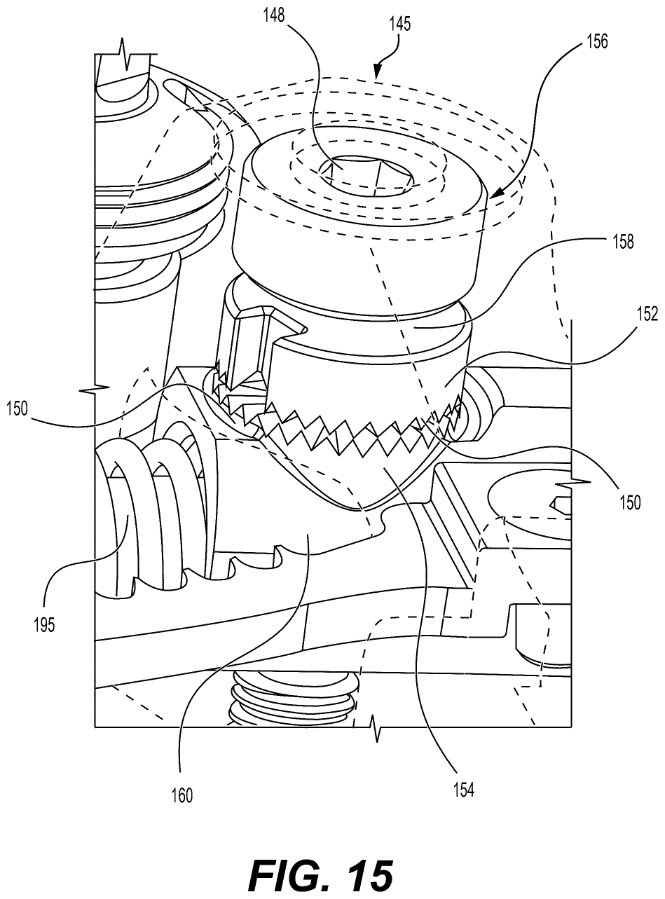

Another implementation of locking mechanism 145 is shown with reference to FIGS. 16-19. In such implementation, locking mechanisms 145 are located and suitably connected at the proximal ends of driveshafts 195 and operate to arrest movement of central arm pinions 191. Because cranial and caudal arms 133 travel in an arced path, driveshafts 195 and corresponding central arm pinions 191 move axially or longitudinally with reference to longitudinal axis L along central arm rack 189 when arms 25 are opened or closed. Accordingly, pinions 191 will rotate about the driveshaft axes when arms 125 are actuated. Locking rotation of pinions 191 will therefore lock the ability of arms 125 to move, articulate, or otherwise be actuated.

To achieve such locking, locking mechanism 145 makes use of a pair of opposing, matable face gears 162 which, in this implementation, are mounting co-axially with the axis of rotation of respective driveshafts 195 which extend transversely to longitudinal axis L. One of face gears 162 is oriented with teeth facing outwardly on an outer transverse surface of the corresponding central arm pinion 191, whereas the second face gear 162 is mounted so that its teeth are opposing such outwardly oriented first face gear.

Second face gear 162 is axially movable into and out of engagement with first face gear 162. In the illustrated implementation, a cam clutch 164 is used to move face gears 162 into and out of engagement, such cam clutch 164 making use of a ramped clutch 162 mounted on central sub assembly 123 and operatively connected to the second of the face gears facing inwardly toward central arm pinions 191. Ramped clutch 166, in turn, is coupled to a lever 168, so as to move ramped clutch 166 so that its ramped portion causes inward, axial translation of the inwardly facing face gear 162 into and out of engagement with the opposing surface of outwardly facing face gear 162.

In this manner, corresponding pinion 191 is arrested or locked which in turn causes motion of the corresponding arm 125 to be selectively locked and unlocked relative to the other arm. In one suitable implementation, as illustrated, the outwardly facing first face gear is integrally formed on an outer transverse surface of a corresponding pinion 191.

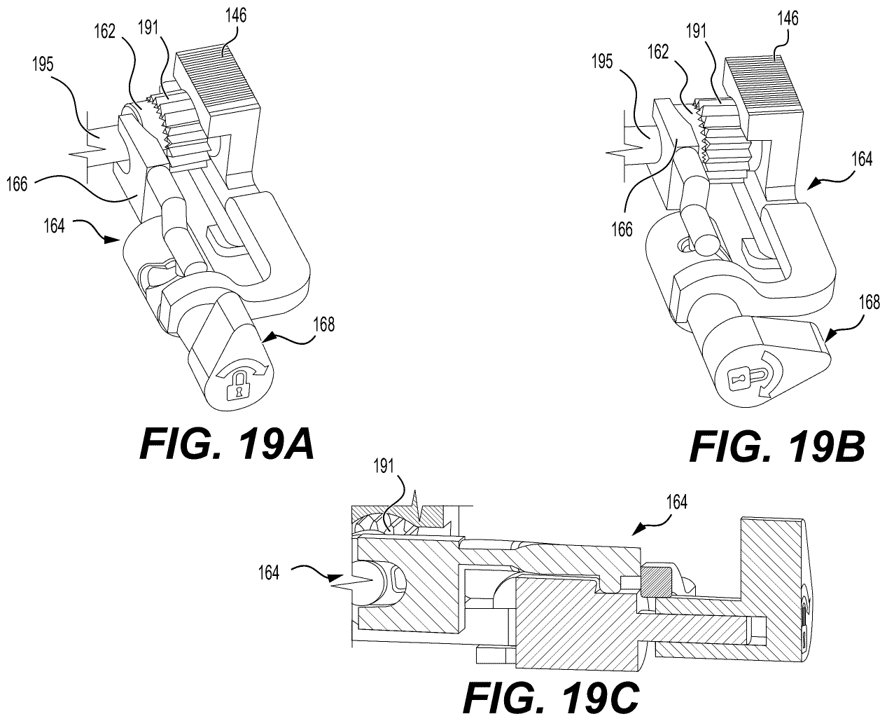

Operation of this embodiment using cam clutch 164 is readily apparent from the foregoing description, and with reference to FIGS. 19A-19C. Lever 168, located at the proximal end of a retractor body housing shown in FIG. 16, may be selectively turned from its open position shown in FIG. 19A, where it has been disengaged, to a closed position shown in FIG. 19B where opposing matable face gears 162 engage each other. Lever 168, when turned, likewise turns a barrel cam as illustrated. Barrel cam is connected to a follower, which is connected to ramped clutch 166, which is driven forward to close cam clutch 164.

In addition, cam clutch 164 includes a member connected to a corresponding one of split bearings 146 so that the components of cam clutch 164 travel along with longitudinal movement of pinions 191 and associated split bearings 146.

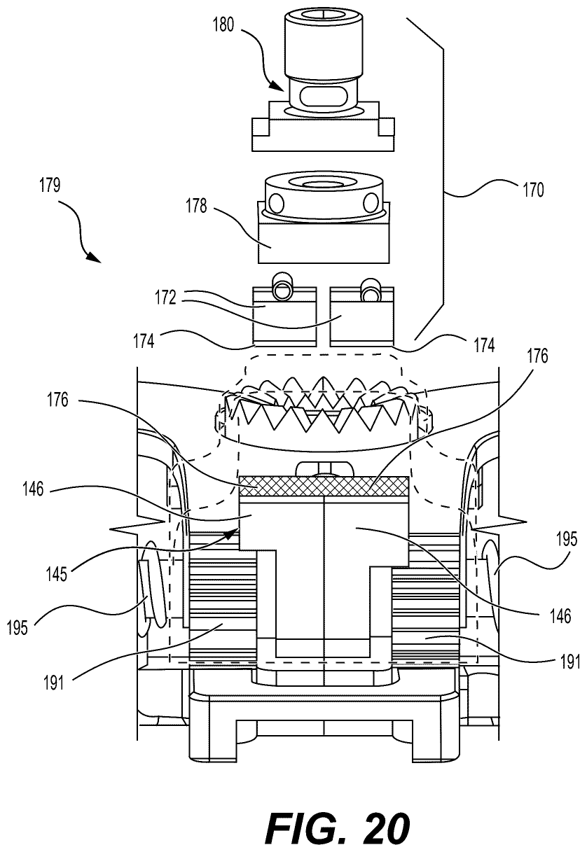

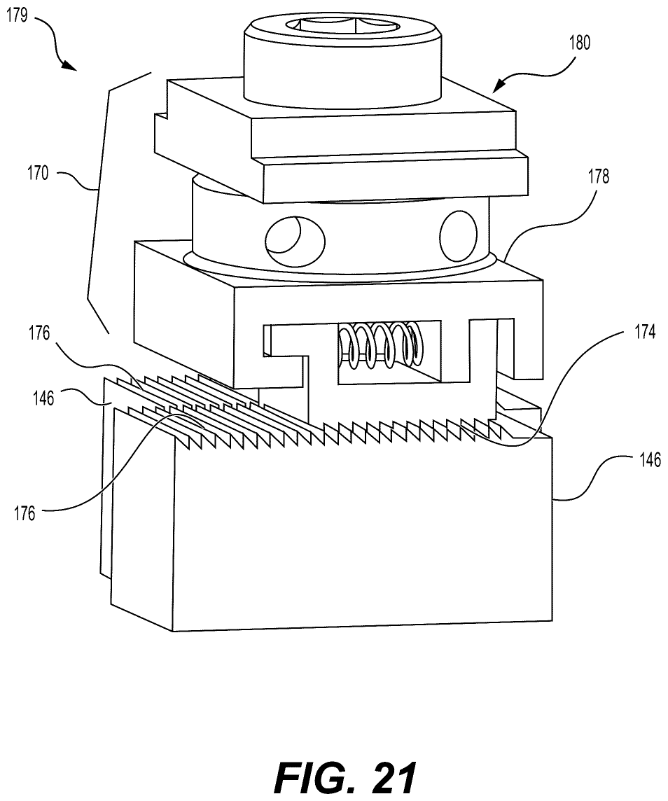

Referring now to FIGS. 20-21, an equal, bilateral retraction mechanism 179, may be used with any suitable lateral retractor, including lateral retractor systems 21, 121. Bilateral retraction mechanism 179 makes use of a certain components and configurations different from that of the equal bilateral retraction mechanism 79 of FIGS. 1-10. Returning to FIGS. 20-21, a knurled clutch 170 is used to couple split bearings 146 to each other, thereby causing motion of one of the arms 125 to induce corresponding motion in the other of the arms. Knurled clutch 170, as shown, includes two, adjacent knurled clutch elements 172 having inner knurled surfaces 174 oriented to engage opposing knurled surfaces 176 on respective split bearings 146. Knurled clutch 170 further includes a moveably mounted clutch housing 178. Housing 178 may be biased to disengage knurled clutch 170 from split bearings 146, and may be selectively actuated to counter the bias and cause engagement on knurled clutch 170.

A clutch engagement mechanism 180 is connected to clutch housing 178. Clutch engagement mechanism 180 may be operated to move the aforementioned clutch housing 178 into and out of engagement with split bearings 146. Accordingly, when clutch engagement mechanism 180 is advanced, such as by means of a rotational set screw, it causes engagement of the knurled clutch elements 172 and the opposing knurled surfaces 176 on split bearings 146 to couple motion of the central arm pinions 191 relative to each other. Coupling the motion of the central arm pinions relative to each other results in equal bilateral movement of arms 125.

It will be further understood that various changes in the details, materials, and arrangements of the parts which have been described and illustrated in order to explain the nature of this invention may be made by those skilled in the art without departing from the scope of the invention as expressed in the claims. One skilled in the art will appreciate that the embodiments discussed above are non-limiting. It will also be appreciated that one or more features of one embodiment may be partially or fully incorporated into one or more other embodiments described herein.

What is claimed is:

1. A lateral retractor system, comprising:
a central subassembly;
a pair of arms having respective proximal and distal portions, the arms connected to the central subassembly relative to a central longitudinal axis and in spaced relation to each other, so that the two arms define a cranial arm and a caudal arm, the two arms pivotably secured at respective articulation axes of the arms, the articulation axes located so that inward movement of the proximal portions toward the central longitudinal axis causes outward movement of the distal portions of the arms to perform lateral retraction on patient anatomy undergoing a spinal procedure;
a central arm movably connected to the central subassembly and selectively advanceable longitudinally in the distal direction;
a cranial-caudal rack connected to the central subassembly and extending arcuately and outwardly on both sides of the central longitudinal axis, the rack terminating in two, opposite rack ends proximate to the proximal portions of the arms, the rack ends having rack teeth formed thereon; and
a pair of locking mechanisms housed on the proximal portions of the arms, respectively, each of the locking mechanisms having a pinion located and configured to rotatably engage the rack teeth;
wherein each of the locking mechanisms comprises a locking foot, the locking foot being selectively actuatable to engage at least one of the rack or a corresponding one of the pinions to lock movement of the corresponding one of the arms, whereby each of the arms may be controlled independently of the other arm.

2. The system of claim 1, further comprising an attachment interface disposed on each of the proximal ends of the arms, the attachment interface configured to attach to at least one of a manually actuatable handle and a driver;
wherein the locking mechanism is housed in a housing connected to the proximal portion of a corresponding arm, the housing having an outer surface and a bore defined therethrough;
wherein the locking mechanism comprises a user depressible button disposed in the bore and accessible from the outer surface of the housing; and
wherein the locking mechanism further comprises a barrel cam rotatably received in the bore and operatively connected to the button, the barrel cam configured to engage the locking foot against the pinion and to disengage the locking foot from the pinion in response to corresponding first and second, respective actuations of the button.

3. The system of claim 2
wherein the pinion is disc-shaped with a cylindrical engagement surface, the pinion having pinion teeth with female portions defined between teeth defined on the cylindrical engagement surface, the pinion teeth configured to engage the rack teeth;
wherein the locking foot terminates in an engagement edge sized to engage a female portion of the pinion teeth between two adjacent ones of the pinion teeth to arrest movement of the pinion relative to the rack, thereby locking the corresponding arm;
wherein each of the arms further comprises a pawl located in the housing and having a distal end extending distally to incrementally engaging the teeth of the rack during corresponding incremental movement of the corresponding arm, the pawl connected at a distance from the locking foot; and
wherein the distance between the pawl and the locking foot is selected to maintain the relative position of the engagement edge of the locking foot in alignment with a corresponding one of the female portions of the pinion at any position of the pinion and the corresponding arm, whereby each of the arms of the system may be selectively locked and unlocked in any relative position.

4. The system of claim 2, wherein the locking mechanism comprises a user-accessible locking screw, the locking screw rotatable relative to the locking foot and connected to the locking foot to engage the pinion in response to rotation in a first direction and to disengage the pinion in response to rotation in a second direction.

5. The system of claim 1, further comprising an equal, bilateral retraction mechanism secured to the central subassembly, the bilateral retraction mechanism having a gear operatively connected to each of the arms, the gears being mounted to the arm to be arrestable by actuation of the bilateral retraction mechanism to connect motion of one of the arms to the other to move the arms equally in response to movement of either of the arms.

6. The system of claim 5, wherein at least one of the gears comprises a face gear, the equal bilateral contraction mechanism comprising a user-actuatable clutch having a clutch plate moveable to engage the face gear, the face gear interconnected to both of the arms to cause equal relative movement thereof.

7. The system of claim 1, wherein the central arm comprises a rack-and-pinion subassembly and an associated central arm pinion to selectively advance and withdraw a distal end of the central arm.

8. The system of claim 7, wherein the central arm pinion is connected to a central arm driveshaft terminating in a handle, the shaft being unconnected to the cranial arm and the caudal arm to permit selective movement of the central arm independently of movement of the cranial and the caudal arms.

9. A lateral retractor system, comprising:
a central subassembly;
a pair of arms having respective proximal and distal portions, the arms connected to the central subassembly relative to a central longitudinal axis and in spaced relation to each other, so that the two arms define a cranial arm and a caudal arm, the two arms pivotably secured at respective articulation axes of the arms, the articulation axes located so that inward movement of the proximal portions toward the central longitudinal axis causes outward movement of the distal portions of the arms to perform lateral retraction on patient anatomy undergoing a spinal procedure;
a central arm movably connected to the central subassembly and selectively advanceable longitudinally in the distal direction;
a cranial-caudal rack connected to the central subassembly and extending arcuately and outwardly on both sides of the central longitudinal axis, the rack terminating in two, opposite rack ends proximate to the proximal portions of the arms, the rack ends having rack teeth formed thereon; and
a pair of locking mechanisms housed on the proximal portions of the arms, respectively, each of the locking mechanisms having a pinion located and configured to rotatably engage the rack teeth; and
an equal, bilateral retraction mechanism secured to the central subassembly, the bilateral retraction mechanism having a gear operatively connected to each of the arms, the gears being mounted to the arm to be arrestable by actuation of the bilateral retraction mechanism to connect motion of one of the arms to the other to move the arms equally in response to movement of either of the arms;
wherein each of the locking mechanisms comprises a locking foot, the locking foot being selectively actuatable to engage a corresponding one of the pinions to lock movement of the corresponding one of the arms, whereby each of the arms may be controlled independently of the other arm;
the system further comprising an attachment interface disposed on each of the proximal ends of the arms, the attachment interface configured to attach to at least one of a manually actuatable handle and a driver;
wherein the locking mechanism is housed in a housing connected to the proximal portion of a corresponding arm, the housing having an outer surface and a bore defined therethrough;
wherein the locking mechanism comprises a user depressible button disposed in the bore and accessible from the outer surface of the housing; and
wherein the locking mechanism further comprises a barrel cam rotatably received in the bore and operatively connected to the button, the barrel cam configured to engage the locking foot against the pinion and to disengage the locking foot from the pinion in response to corresponding first and second, respective actuations of the button;
wherein the central arm comprises a rack-and-pinion subassembly and an associated central arm pinion to selectively advance and withdraw a distal end of the central arm; and
wherein the central arm pinion is connected to a central arm driveshaft terminating in a handle, the shaft being unconnected to the cranial arm and the caudal arm to permit selective movement of the central arm independently of movement of the cranial and the caudal arms.

10. A lateral retractor system, comprising:
a central subassembly;
a pair of arms having respective proximal and distal portions, the arms connected to the central subassembly relative to a central longitudinal axis and in spaced relation to each other, so that the two arms define a cranial arm and a caudal arm, the two arms pivotably secured at respective articulation axes of the arms, the articulation axes located so that inward movement of the proximal portions toward the central longitudinal axis causes outward movement of the distal portions of the arms to perform lateral retraction on patient anatomy undergoing a spinal procedure;
a central arm having opposite lateral sides and opposite proximal and distal ends, the central arm movably connected to the central subassembly and selectively advanceable and retractable longitudinally to move the distal end relative to a patient anatomy undergoing a spinal procedure;
a central arm rack disposed on the central arm and extending in the longitudinal direction;
a pair of driveshafts extending transversely from the sides of the central arm in relation to the longitudinal axis to define respective outer and inner driveshaft ends, the inner driveshaft ends rotatably connected to the central arm rack at separate, respective locations through central arm pinions to selectively drive the central arm distally or proximally using either of the driveshafts;
a pair of split bearings coupling the inner driveshaft ends to the central subassembly;
wherein the outer driveshaft ends are pivotally connected at pivot points on the proximal portions of the arms, whereby movement of one of the arms relative to the other of the arms translates the corresponding bearing relative to the other bearing;
a pair of locking mechanisms operatively connected to the pair of arms, each of the locking mechanisms configured to lock a corresponding one of the pivot points in response to user actuation of the locking mechanism to arrest movement of the corresponding one of the arms independently of the other of the arms.

11. The system of claim 10
wherein each of the locking mechanisms comprises a pair of opposing, matable face gears axially mounted relative to the axis of the pivot point;
wherein the first face gear comprises a free gear keyed rotationally to the arm to be free to rotate about the pivot point when the arm pivots about the pivot point;
wherein the second face gear comprises a fixed gear non rotatably mounted relative to the pivot point;
wherein the opposing surfaces of the gears are mounted to be translatable between first and second positions, the first position corresponding to a disengaged position in which the opposing surfaces are in spaced relationship, and the second position corresponding to an engaged position in which the opposing surfaces are mated; and
wherein the locking mechanism further includes a clutch to selectively move the pair of gears between the disengaged position and the engaged position.

12. The system of claim 11, wherein the clutch comprises a threaded set screw rotatable in a first direction to move the face gears relatively toward each other to engage the locking mechanism, and rotatable in a second direction to move the face gears relatively away from each other to disengage the locking mechanism.

13. The system of claim 10, wherein the pair of locking mechanisms are housed in bushings surrounding the driveshafts on respective proximal portions of the arms.

14. The system of claim 10,
wherein each of the locking mechanisms comprises a pair of opposing matable face gears axially mounted relative to a respective one of the transversely oriented driveshafts, the first face gear oriented outwardly on an outer, transverse surface of the pinion and rotatable therewith, the second face gear mounted to be axially movable into and out of engagement with the first face gear;
wherein the locking mechanism further including a cam clutch comprising a ramped clutch mounted on the central subassembly and operatively connected to the second face gear, the cam clutch including a lever operatively connected to move the ramped clutch to translate the second face gear into and out of engagement with the first face gear, whereby the motion of the corresponding one of the arms is selectively locked and unlocked relative to the other of the arms.

15. The system of claim 14, wherein the first, face gear is integrally formed on the outer, transverse surface of the pinion.

16. The system of claim 10, further comprising:
an equal, bilateral retraction mechanism including a knurled clutch to couple the split bearings to each other, thereby causing the motion of one of the arms to induce corresponding motion of the other of the arms;
the knurled clutch including two, adjacent knurled clutch elements with inner knurled surfaces to engage opposing knurled surfaces on respective first and second split bearings, the knurled clutch further including a moveably mounted clutch housing, the clutch housing biased to disengage the knurled clutch from the split bearings; and
a clutch engagement mechanism connected to the central assembly and operable to move the clutch housing to engage the split bearings with the knurled clutch elements to couple motion of one of the pinions to the other of the pinions, whereby equal bilateral retraction of the arms occurs.

17. The system of claim 10, further comprising an attachment interface disposed on each of the proximal ends of the arms, the attachment interface configured to attach to at least one of a manually actuatable handle and a driver.

18. The system of claim 10, further comprising:
an equal, bilateral retraction mechanism including a knurled clutch to couple the split bearings to each other, thereby causing the motion of one of the arms to induce corresponding motion of the other of the arms;
wherein the knurled clutch includes two, adjacent knurled clutch elements with inner knurled surfaces to engage opposing knurled surfaces on respective first and second split bearings, the knurled clutch further including a moveably mounted clutch housing, the clutch housing biased to disengage the knurled clutch from the split bearings; and
a clutch engagement mechanism connected to the central assembly and operable to move the clutch housing to engage the split bearings with the knurled clutch elements to couple motion of one of the pinions to the other of the pinions, whereby equal bilateral retraction of the arms occurs;
wherein each of the locking mechanisms comprises a pair of opposing, matable face gears axially mounted relative to the axis of the pivot point;
wherein the first face gear comprises a free gear keyed rotationally to the arm to be free to rotate about the pivot point when the arm pivots about the pivot point;
wherein the second face gear comprises a fixed gear non rotatably mounted relative to the pivot point;
wherein the opposing surfaces of the gears are mounted to be translatable between first and second positions, the first position corresponding to a disengaged position in which the opposing surfaces are in spaced relationship, and the second position corresponding to an engaged position in which the opposing surfaces are mated; and
wherein the locking mechanism further includes a clutch to selectively move the pair of gears between the disengaged position and the engaged position;
wherein the pair of locking mechanisms are housed in bushings surrounding the driveshafts on respective proximal portions of the arms.

19. The system of claim 10, further comprising:
an equal, bilateral retraction mechanism including a knurled clutch to couple the split bearings to each other, thereby causing the motion of one of the arms to induce corresponding motion of the other of the arms;
wherein the knurled clutch includes two, adjacent knurled clutch elements with inner knurled surfaces to engage opposing knurled surfaces on respective first and second split bearings, the knurled clutch further including a moveably mounted clutch housing, the clutch housing biased to disengage the knurled clutch from the split bearings; and
a clutch engagement mechanism connected to the central assembly and operable to move the clutch housing to engage the split bearings with the knurled clutch elements to couple motion of one of the pinions to the other of the pinions, whereby equal bilateral retraction of the arms occurs;
wherein each of the locking mechanisms comprises a pair of opposing matable face gears axially mounted relative to a respective one of the transversely oriented driveshafts, the first face gear oriented outwardly on an outer, transverse surface of the pinion and rotatable therewith, the second face gear mounted to be axially movable into and out of engagement with the first face gear;
wherein the locking mechanism further including a cam clutch comprising a ramped clutch mounted on the central subassembly and operatively connected to the second face gear, the cam clutch including a lever operatively connected to move the ramped clutch to translate the second face gear into and out of engagement with the first face gear, whereby the motion of the corresponding one of the arms is selectively locked and unlocked relative to the other of the arms.
